(19) United States
(12) Patent Application Publication
(10) Pub. No.: US 2007/0166735 A1
(43) Pub. Date: Jul. 19, 2007

(54) METHODS OF IN VITRO PROPAGATION AND DETECTION OF INFECTIOUS PRION

(75) Inventor: ALAN YOUNG, Brookings, SD (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SOUTH DAKOTA STATE UNIVERSITY, Brookings, SD (US)

(21) Appl. No.: 11/566,094

(22) Filed: Dec. 1, 2006

Related U.S. Application Data
(60) Provisional application No. 60/748,494, filed on Dec. 8, 2005.

(51) Int. Cl. C12Q 1/68; G01N 33/53

(52) U.S. Cl. 435/6; 435/7.1

(57) ABSTRACT

Methods for the in vitro propagation of infectious prions (PrP^{Sc}) are provided. Follicular dendritic cells (FDCs) are cultured with B cells and infected with prions. Methods of detecting infectious prions (PrP^{Sc}) in an animal or human are also provided. Peripheral blood B cells are collected from an animal or human suspected of being infected with infections prions, cultured with follicular dendritic cells, and the presence of infectious prions is detected.

### CROSS-REFERENCE TO RELATED APPLICATION

This application claims the benefit of earlier filed U.S. Provisional Application Ser. No. 60/748,494, filed Dec. 8, 2005, which is incorporated herein by reference.

### BACKGROUND

The invention relates to a method for in vitro propagation of infectious prion proteins, and methods of detecting prion disease in fluid, tissue or cellular samples.

A prion is a transmissible particle devoid of nucleic acid. The most notable prion diseases are Bovine Spongiform Encephalopathy (BSE), Scrapic of Sheep, Chronic Wasting Disease (CWD) in cervids (deer, elk, and moose), and Creutzfeldt-Jakob Disease (CJD) of humans. Prions appear to be composed exclusively of a modified isoform of prion protein (PrP) called PrP^{Sc}. The normal cellular PrP (called PrP^{C}) is converted into infectious PrP^{Sc} through a post-translational process. During this process, the structure of PrP^{C} is altered and is accompanied by changes in the physiochemical properties of PrP. Prions are believed to cause disease through the ability of a conformationally-altered protein (PrP^{Sc}) to induce the refolding of a native cellular protein (PrP^{c}) to the pathogenic form. It is the proliferation of this protein conversion reaction which ultimately results in the formation of the characteristic spongiform plaques which form in the brains of infected individuals.

In general, natural transmission of prion diseases is believed to occur through ingestion of infectious material, although accidental transmission has occurred in humans through transplantation of blood and solid organs, as well as through contaminated surgical instruments. Transmission of CWD in the wild is believed to occur as a result of either direct blood-to-blood contact, or oral ingestion of prion infected material, although there is evidence to suggest that CWD may be more prone to horizontal transmission than other prion disorders suggesting additional reservoirs such as urine or feces. The pathogenic prion proteins are transported either across the gut wall and into the intestinal immune system or directly into the tonsils during ingestion, where they infect the regional immune system. These infectious prions replicate within active areas of migratory B cell proliferation directed by stationary Follicular Dendritic Cells (FDCs).

Infection of the brain then occurs as a result of the prion replication traveling up the regional nerves. Areas of chronic inflammation, particularly associated with FDC-B cell accumulations, also result in prion propagation. While the means whereby infectious prion protein “seeds” these areas of lymphoid accumulation is unclear, the most direct route for infection of these follicles is via migratory B cells.

A primary difficulty in diagnosis of these diseases has been an inability to expand the low levels of infectious prion in infected but asymptomatic individuals to a level detectable by current assays. Although it is known that blood can transmit disease from infected individuals, no current assays are capable of detecting PrP^{Sc} in blood. In contrast, diagnosis generally relies upon analysis of histological sections of brain and lymph node post-mortem. One successful antemortem test for scrapie relies upon detection of PrP^{sc} in lymphoid tissue of the sheep eyelid. While many cell types appear to express the normal cellular form of prion protein, only a select number appear to serve as reservoirs of infections prion protein during disease. In addition to neural cells, only follicular dendritic cells (FDC) in the germinal centers of lymph nodes have been shown to be absolutely essential for normal development of prion disease. While FDCs are believed to be the first affected cell type during oral infection, it is important to recall that even during experimental intracerebral infection, FDCs in select lymph nodes (retropharyngeal and mesenteric) still appear to concentrate and proliferate PrP^{Sc}. In fact, normal oral infection is believed to rely upon transmission from PrP^{Sc}-laden FDCs within mesenteric lymph nodes to the brain via peripheral nerves. This ability of FDCs to concentrate PrP^{Sc} appears to be related to their ability to bind and concentrate foreign proteins complexed with complement components.

It has been demonstrated in experimental studies that the earliest recognizable source of infectious prions in cattle is the ileum, containing ileal Peyer's Patches. This tissue remains infective throughout incubation, as the disease progresses through the neuronal tissues. Bovine Spongiform Encephalitis (BSE) is unique among the transmissible spongiform Encephalopathies (TSE) in its apparent ability to cross species barriers. Specifically, consumption of BSE-affected beef is believed to have resulted in the development of a variant form of Creutzfeld Jakob Disease in humans. While there are currently only 156 reported human cases as a result of the “BSE Epidemic” in Europe during the late 20^{th} century, recent data may indicate that human prion diseases may have extended incubation period exceeding 40 years duration. Since large-scale testing has been instituted for BSE, it has become evident that there exist both the traditional infectious form of BSE, as well as a novel form generally referred to as “atypical BSE”. It is significant that both US BSE cases identified to date are of this atypical form. While the significance of this atypical BSE remains unclear, studies have clearly demonstrated that both forms of BSE are potentially infectious. It is also significant that experimental studies have demonstrated that infectious prions are present in the ileal tissues of cattle within several months of infection, long before the appearance of lesions or histologically-detectable levels of prions in the brain. It is therefore crucial to develop a screening assay for BSE capable of detecting this early stage disease in living cattle.

The biochemical nature of PrP^{Sc} appears to be highly species specific. More specifically, individual strains of prion diseases (i.e., scrapie, Chronic Wasting Disease) appear to promote the formation of unique ratios of non, mono, and di-glycosylated PrP^{Sc} in susceptible hosts. This specificity appears to be further reflected in differences depending upon the species studied. It is therefore imperative to develop species-specific methods for the culture of PrP^{Sc} which can be used to expand small amounts of PrP^{Sc} for diagnostics and research use.

An ideal diagnostic technique would therefore involve expansion of the small number of prions associated either with peripheral blood B cells or free in tissue fluids, which can then be detected using conventional methods.

### SUMMARY

The present invention provides a method for the in vitro propagation of infectious prions (PrP^{Sc}). The method involves providing a culture of follicular dendritic cells (FDC), adding sample materials including but not limited to serum, cerebrospinal fluid, urine, saliva, or peripheral B cells to the FDC culture to stimulate expansion of infectious prions. As natural sites of PrPSc concentration in diseased individuals, FDCs in vitro provide a method to both capture and replicate the small amounts of infectious PrPSc in diagnostic samples to detectable levels.

In another embodiment, a method of detecting infectious prions (PrP^{Sc}) in an animal or human is provided. The detection method involves collecting peripheral blood B cells from an animal or human suspected of being infected with infections prions, co-culturing the B cells with cultured follicular dendritic cells, and detecting infectious prions using a specific binding assay. In some embodiments, the specific binding assay is an immunological assay, such as immunohistochemistry or Western blots.

In some embodiments, the animal is an ovine, and the immunological assay involves an antibody specific for scrapie. In other embodiments, the animal is a cervid, and the immunological assay involves an antibody specific for Chronic Wasting Disease (CWD). In still further embodiments, the method is for detection of infectious prions in a human, and the immunological assay involves an antibody that binds human prion protein (PrP). In a final embodiment, the method is for detection of infectious prions in cattle, and the immunological assay involves an antibody that binds bovine prion protein.

In an additional method for detecting infectious prions (PrP^{Sc}) in an animal or human, a fluid, cellular or tissue sample is obtained from an animal or human suspected of being infected with infections prions. The sample is added to a culture of follicular dendritic cells, and the cells are cultured. Infectious prions are then detected in the culture by a specific binding assay. In some embodiments, the culture of follicular dendritic cells includes B-cells. In further embodiments, the specific binding assay is an immunological assay, such as immunohistochemistry or Western blot. The sample can be blood, brain, spleen, spinal fluid, lymph nodes, urine, saliva, feces, or tonsils.

In a further embodiment, the invention provides a method for the in vitro propagation of infectious prions (PrP^{Sc}) in which an animal susceptible to a prion disorder is selected. Lymph node cells are obtained from the animal, and those lymph node cells that bind antibodies specific for FDCs are selected. The resulting cells are cultured, and cells from the culture that bind antibodies specific for prion protein are selected. The selected cells are then infected with infectious prions and cultured to define the assays described below. In one embodiment, the step of selecting an animal involves selecting an animal genetically susceptible to a prion disorder. In some embodiments, the animal is an ovine and said prion disorder is scrapie. In other embodiments, the animal is a cervid and the prion disorder is Chronic Wasting Disease (CWD), the animal is a bovine and the prion disorder is CWD, and in the case of humans the prion disorder is CJD.

In a still further embodiment, the invention provides a method for detecting, and optionally quantifying, prion in a biological sample. The method involves contacting the biological sample with a culture of FDCs and B cells under conditions that allow the infection thereof, and detecting infection or non-infection of the cultured cells. The presence of infection is indicative of prion in the sample. In some embodiments, the presence of infection is detected by an immunological assay. Samples can include blood, lymph node, and brain. In some embodiments, the mixed culture of FDCs and B cells include cells isolated from an animal genetically susceptible to prion disease.

In another embodiment, a kit is provided for detecting infectious prions (PrP^{Sc}) in a biological sample. The kit includes cultured follicular dendritic cells (FDCs) and antibodies specific for infectious prions (PrP^{Sc}). The kit can also include B cells co-cultured with the FDCs. In some embodiments, the FDCs are cervid and the antibodies specifically bind Chronic Wasting Disease (CWD). In other embodiments, the FDCs are ovine and the antibodies specifically bind sheep Scrapie.

### BRIEF DESCRIPTION OF THE DRAWINGS

FIG. 1 is an illustration of the FDC culture model.

FIG. 2 shows the immunohistochemistry of ileal Peyer's Patches and retropharyngeal lymph node tissues.

FIG. 3 shows flow cytometric analysis of phenotype of cultured ovine FDCs.

FIG. 4 shows flow cytometric analysis of cultured FDCs three and 34 months after initial culture.

FIG. 5 shows the morphology of cervid FDCs following infection with CWD-positive brain homogenate.

FIG. 6 is a bar graph showing cultured FDCs support the proliferation of B cells in vitro.

FIG. 7 is a bar graph showing cultured FDCs support the proliferation of B cells in vitro.

FIG. 8 is a bar graph showing cultured FDCs support the proliferation of B cells in vitro.

FIG. 9 shows PrP^{Sc}in the cytoplasm of FDCs infected in vitro.

FIG. 10 is a slot blot showing PrP^{Sc} in FDCs infected in vitro.

FIGS. 11A and 11 show the disproportionate representation of B-1 cells in PrP^{Sc} infected animals.

FIG. 12 is a graph showing the reduction in PrP^{C} expression on B-1 cells during scrapie progression.

FIG. 13 is a graph showing reduction in B-cell output from scrapie-inoculated lymph nodes.

FIG. 14 shows the transport of prions by migratory B cells.

FIG. 15 is a flow chart showing the isolation and Western blot analysis of PrP^{CWD}.

FIG. 16 is a Western blot of sheep FDCs infected with scrapie.

FIG. 17 is a Western blot of Peyer's Patch-derived elk FDCs infected with CWD-positive brain homogenate.

FIG. 18 is a Western blot of mesenteric lymph node-derived elk FDCs infected with CWD-positive brain homogenate.

FIG. 19 is a Western blot of retropharyngeal lymph node-derived elk FDCs infected with CWD-positive brain homogenate.

FIGS. 20A and 20B are Western blots of cattle FDCs infected with sheep scrapie.

### DETAILED DESCRIPTION

The present invention provides an in vitro replication system for prions based on the replication of infectious prions in germinal centers during infection. The system has two distinct advantages for the early detection of low levels of infectious prions:

a) Migratory B cells may be directly harvested from the blood of animals, and tested for the presence of infectious prions by plating on cultured FDCs.

b) Given that FDCs are specialized cells whose primary function is to concentrate rare molecules to stimulate B cells, the system is pre-optimized by nature to collect, concentrate, and replicate infectious prions.

As used herein, “propagation” or “replication” of the prion in a cell culture means that, after infection, or infestation, of at least one cell of the starting cell culture or of the starting cell line, the infectious capacity of the prion is conserved in the derived cells, i.e. the cells resulting from subcultures.

### EXAMPLES

The following examples are intended only to further illustrate the invention and are not intended to limit the scope of the invention which is defined by the claims.

### Example 1

It has previously been demonstrated that susceptibility to prion disorders is genetically determined. This is most clearly illustrated in the case of sheep scrapie and elk CWD, where distinct amino acids in the coding region of the prion gene regulate susceptibility to CWD infection. With respect to elk, the presence of a Methionine residue at position 132 of the prion gene is a recessive determinant of susceptibility. The situation in deer is less clear, although it appears to be linked to at least 4 distinct loci. Animals genetically susceptible to CWD were first identified. Once identified, these animals were used as donors to establish FDC cultures. Blood samples from 10 elk and 10 white-tail deer were obtained from a breeder for genetic sequencing of the prion gene. Results are presented in Table 1.

TABLE 1
Predicted Susceptibility of White Tail Deer and Elk
to CWD screened for production of FDC cultures.

| WHITE TAIL DEER | | ELK | |
|---|---|---|---|
| Animal # | Susceptibility to CWD | Animal # | Susceptibility to CWD |
| G1 | Medium | *Y107 | Low |
| G7 | Medium | *Y22 | High |
| *O14 | Low | O17 | High |
| *W20 | High | G16 | High |
| W27 | Medium | *G9 | High |
| *Y3 | High | 39J | Low |
| Y11 | Medium | 24 | High |
| Y44 | High | 8KY | High |
| Y71 | High | 4K | High |
| W3 | Medium | G2 | High |

(*Animals selected as donors for production of FDC cultures).

Briefly, the majority of available elk appeared to be homozygous for Methionine at codon 132, denoting susceptibility. The situation was less defined in white-tail deer. 3 animals were identified that were genetically highly susceptible to CWD. 1 elk was identified as genetically resistant to CWD, and 1 deer identified as being of lesser susceptibility to CWD. These animals were obtained from the farm for production of FDC cultures. It should be noted that within the tested elk population, no animals homozygous for the resistance-associated Leucine at codon 132 were identified. This supports the observation that the CWD resistant phenotype is rare within the farmed cervid population, further illustrating the need for a highly-sensitive ante mortem test for CWD.

### Example 2

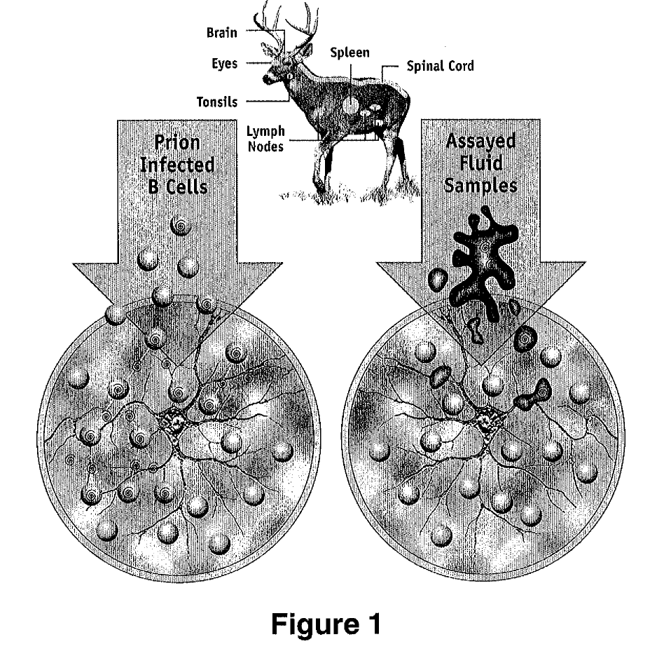

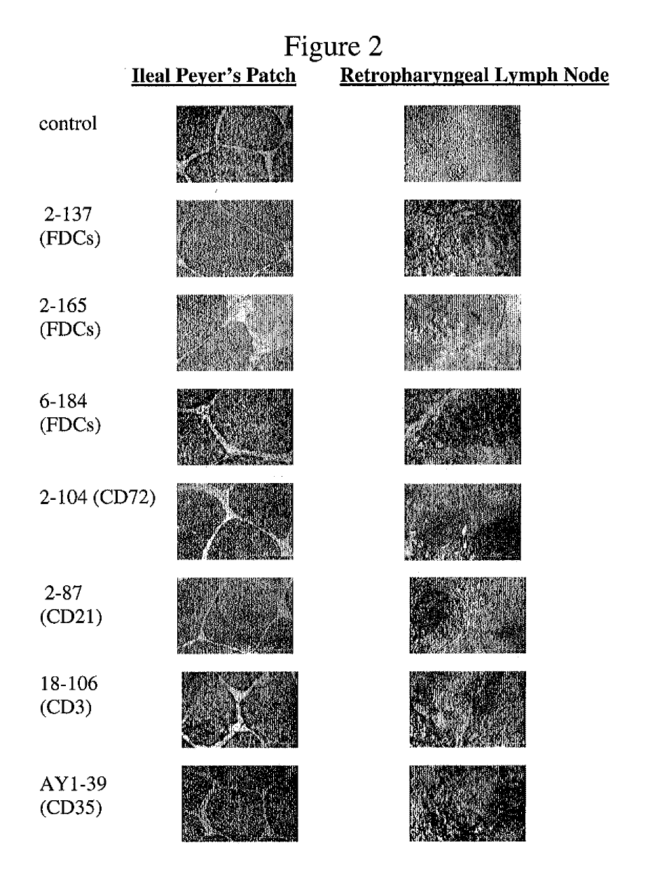

Primary cultures of deer and elk FDCs were isolated from lymph nodes of genetically susceptible animals. Animals selected according to Example 1 were procured from a regional farm, anesthetized using ketamine/xylazine, and sacrificed by electrocution according to standard procedure of the South Dakota Veterinary Diagnostic Laboratory. Whole retropharyngeal and mesenteric lymph nodes were then obtained from the freshly killed animals and processed according to standard procedures to produce a single-cell suspension. Cells were then incubated for 15 minutes with antibodies previously identified as reacting specifically with FDCs, followed by secondary staining with magnetic-bead conjugated goat-anti-mouse commercial antibody. These cells were then selected using an AutoMACS and the positive cells cultured in rich tissue culture media containing 10% fetal calf serum. Their identity was confirmed by cellular surface markers, morphology, and proliferation capability. FIG. 2 shows immunohistochemical staining of ileal Peyer's Patches and Retropharyngeal lymph nodes from a 3 month old lamb. Cells were fed at 3-4 day intervals with new media, and split when initial wells reached confluence. After the 3rd passage, cells were trypsinized and reacted with antibodies against surface prion protein (6H4, Prionics AG, Switzerland). All clones expressed significant levels of prion protein, necessary to support propagation of prions in vitro. See FIG. 1.

### Example 3

The utility of the cells obtained in Example 2 to support prion propagation in vitro was defined. The time-intensive nature of these experiments had significant effects on the final testing of the efficacy of these cells to support prion propagation. Specifically, FDCs are extremely slowly growing cells, and once confluent cultures are achieved, further infection with prions requires a minimum of 2-4 weeks to be definitive.

The following results were obtained using FDC-B cell cultures infected with sheep scrapie. The culture method is a refinement of previous reports used to establish stable FDC lines from cattle and humans. A panel of monoclonal antibodies was used in conjunction with magnetic separation to purify follicular dendritic cells from lymph node suspensions and ileal Peyer's Patches. The antibodies used for the isolation and characterization of ovine FDCs are shown in Table 2. Antibodies 2-137, 2-165, and 6-184 were used for the isolation of FDCs from lymphoid tissues. Antibody 32A16 is deposited with the European Cell and Culture Collection (ECACC), and antibodies 3C10, E2/51, and M2/61 are deposited in the ATCC.

TABLE 2

| | | | Cross-React | Cellular |
|---|---|---|---|---|
| Antibody | Isotype | Target | to Deer/Elk? | Expression |
| 2-165-4 | IgM | FDCs | Yes | FDCs |
| 6-184A1 | IgG2a | FDCs | Yes | FDCs |
| 2-137 | IgM | FDCs | Yes | FDCs |
| 2-87 | IgG1 | CD21 | Yes | B cells, FDCs |
| 2-54 | IgM | CD21 | Yes | B cells, FDCs |
| 6H4 | IgG1 | PrP | Yes | Ubiquitous |
| (Prionics) | | | | |
| AYI-39 | | CD35 | | Erythrocytes, |
| | | | | neutrophils, |
| | | | | monocytes, |
| | | | | eosinophils, B |
| | | | | cells, FDCs |
| M2/61 | | CD40 | | B cells, FDCs, |
| | | | | endothelial cells |
| E2/51 | | CD154/ | | Activated T cells, |
| | | CD40L | | FDCs |
| 2-104 | | CD72 | | B cells |
| 12-5-4 | IgG1 | CD11b | | Monocytes, |
| | | | | DCs, FDCs |
| 3C10 | IgG1 | CD14 | | Monocytes/ |
| | | | | macrophages |
| 1-88 | IgG1 | CD85 | | B cells |
| 32A16 | IgG1 | MHC-II | | CDs, B cells, |
| | | | | monocytes/ |
| | | | | macrophages |

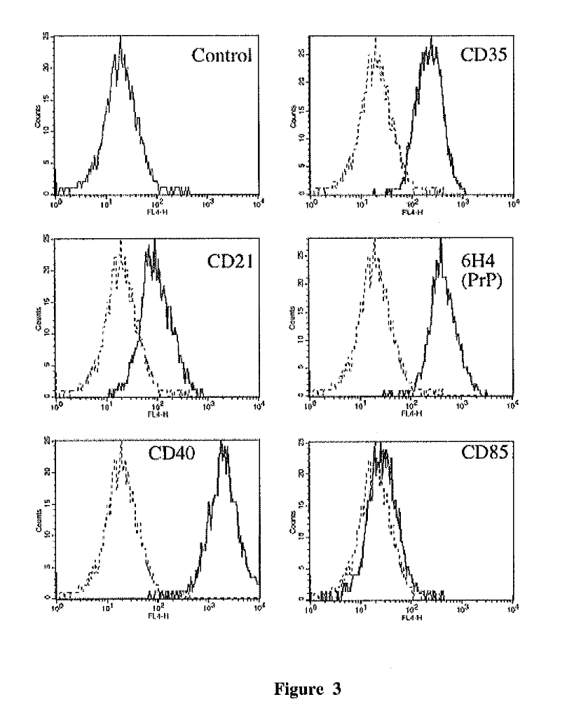

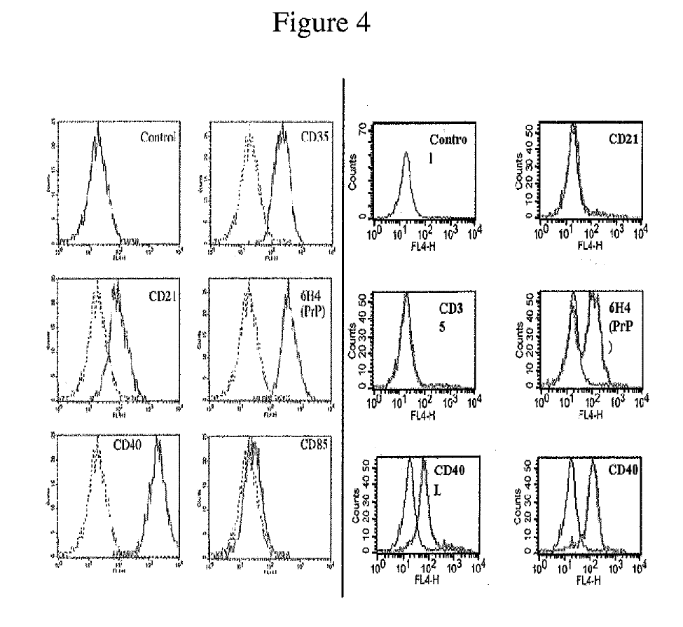

The cells morphologically resemble FDCs in culture, and express the cell surface markers CD21, CD40, and CD35 which are distinct for FDCs but not fibroblasts. See FIG. 3, showing flow cytometric analysis of the phenotype of cultured ovine FDCs. Control staining is shown in dotted lines. The FDCs are shown to express CD35, CD21, PrP, and CD40 but not the B cell marker CD85. Most importantly, these cells continue to express high levels of PrP^{C}, which may be required for conversion of PrP^{C} to PrP^{Sc} in vitro. FIG. 4 shows flow cytometric analysis of the cultured FDCs three months (left) and 34 months (right) following initial culture. While CD21 and CD35 have been downregulated, CD40, CD40L, and PrP^{C} continue to be expressed.

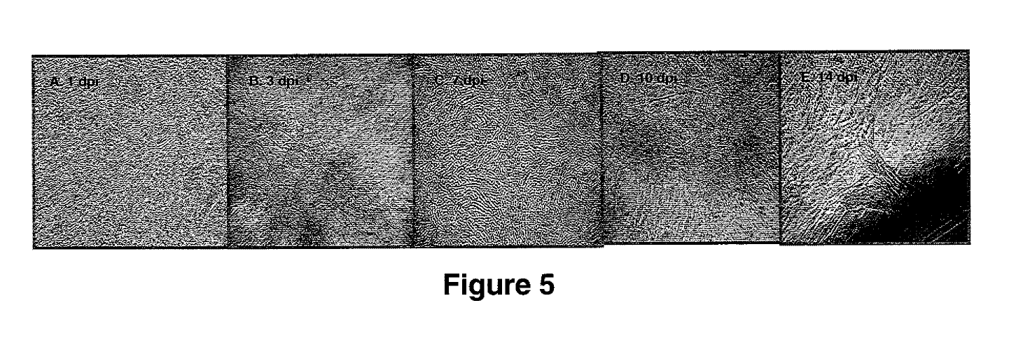

FIG. 5 shows the morphology of cervid FDCs following infection with CWD-positive brain homogenate. Cells were infected on day 0 with 100 μl of 10% infectious brain homogenate. The cells and supernatants (photo A) were collected 24 hours after infection. These cell lines were characterized by their large size, coupled with an extremely slow rate of cell division. In culture, adherent cells displayed typical dendritic morphology consistent with an FDC phenotype. Surprisingly, these cells have remained in culture for over 2 years, in the absence of transformation, by being fed at 3-4 day intervals and split to new flasks every 2-3 weeks.

Several lines were selected for further characterization. While these cells morphologically resembled FDCs in culture, it was important to further define their surface expression of FDC-associated cell surface proteins. FDC cultures were trypsinized, and labeled with antibodies directed against CD21, CD35, CD40, PrPc, and CD85. Notably, FDC cultures expressed high levels of the lineage-related proteins CD21, CD35, and CD40 (FIG. 3). More importantly, cultured FDC lines expressed levels of PrP^{c} significantly higher than those observed by B cells, and failed to express the B-cell antigen CD85. The phenotype of the cultured cell lines was consistent with that of FDCs.

In addition to cell line 6A, the following sheep FDC lines have been developed:
JFDC2-IPP 2-65
JFDC2 RPLN 2-165
JFDC2 IPP 6-184
JFDC2 RPLN 6-184
JFDC2 IPP 2-137
JFDC2 RPLN 2-137
   The cell lines are named according
   to the antibody used for isolation (2-165, 6-184, 2-137) and the tissue from which they were prepared (RPLN=Retropharyngeal Lymph Node; IPP=Ileal Peyer's Patch). Cell line 6A was isolated from the Retropharyngeal lymph node of a susceptible sheep.

The following 12 elk and 1 deer FDC lines have been developed:

| | | | |
|---|---|---|---|
| Elk | Y107 | Mes | 6-184 |
| Elk | Y107 | Mes | 2-137 |
| Elk | Y107 | RP | 6-184 |
| Elk | Y107 | RP | 2-137 |
| Elk | G9 | Mes | 6-184 |
| Elk | G9 | Mes | 2-137 |
| Elk | G9 | RP | 6-184 |
| Elk | G9 | RP | 2-137 |
| Elk | Y2G | Mes | 6-184 |
| Elk | Y2G | Mes | 2-137 |
| Elk | Y2G | RP | 6-184 |
| Elk | Y2G | RP | 2-137 |
| Deer | Y3 | RP | 6-184 |

Mes = Mesenteric Lymph Node
RP = Retropharyngeal Lymph Node.

The following cattle FDC lines have been developed:
NCIPP (normal cow, ileal Peyer's patch line)
HIPP (prion knockout animal, ileal Peyer's patch line)
NCRPLN (Normal cow, Retropharyngeal Lymph node line)
HRPLN (Prion knockout cow, Retropharyngeal lymph node).

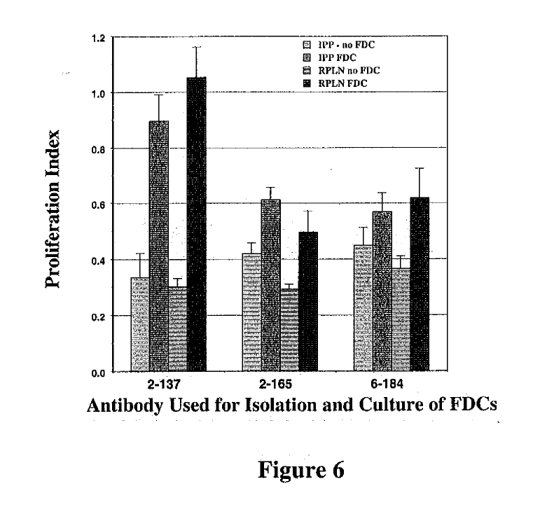

Cultured FDC lines support the proliferation of B cells in vitro (FIG. 6). B cells were isolated by negative magnetic selection, and plated on FDC lines originally isolated from ileal Peyer's Patch (IPP) or retropharyngeal lymph node (RPLN) using monoclonal antibodies (mAbs) 2-137, 2-165, or 6-184. Three days following initiation of culture, a commercial BrdU-based ELISA was used to assess proliferation of the B cells. While B cells alone failed to divide in culture, all FDC lines supported increased B cell growth. Those FDCs isolated using mAb 2-137 appeared to be the most effective at supporting B cell proliferation in vitro.

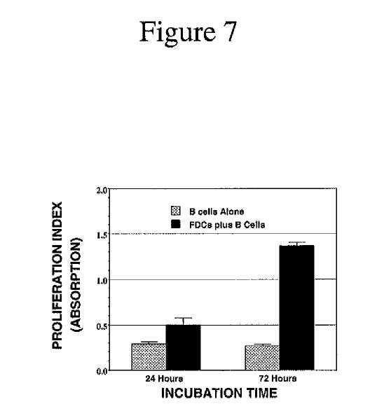

The primary function of FDCs is to present appropriate antigen complexes and additional signals to support B cell replication independent of major histocompatibility complex (MHC) restriction. The ability of the cell lines to support ovine B cell proliferation in vitro was determined. FDC cell lines were seeded onto flat-bottom 96 well cell culture plates. Peripheral blood mononuclear cells were collected from uninfected sheep, and purified by density-gradient separation. B cells were then purified by negative selection using the AutoMACS, counted, and cells were plated into 96-well plates in the presence or absence of confluent FDCs. B cells were then incubated for 24 or 72 hours prior to analysis with a commercial BrdU-based proliferation assay (FIG. 7). While B cells alone did not actively proliferate in the absence of mitogen, the addition of FDC monolayers significantly promoted proliferation of peripheral blood B cells at 24 and 72 hours post co-cultivation. Also, the morphology of the FDC lines dramatically changed in the presence of B cells. These data indicate that ovine FDC lines are capable of supporting B cell proliferation in vitro.

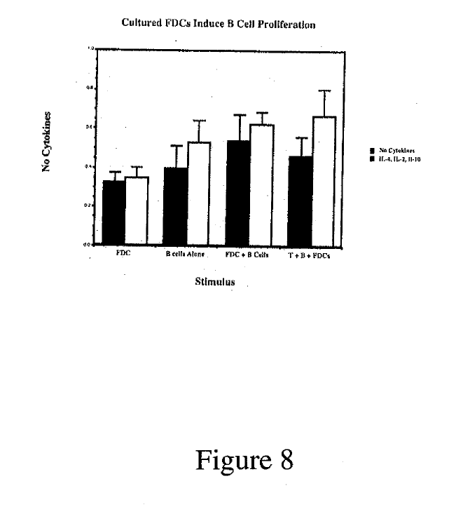

Cervid FDCs have been cultured according to Example 2. These cells also express high levels of PrP^{C}. We have confirmed that these ovine cells support B cell proliferation in vitro as previously described in other systems, functionally identifying them as FDCs. See FIG. 8, which shows tat cultured FDCs support B cell proliferation in vitro. Peripheral blood B cells were sorted by MACS technology and plated on cultured FDCs in the presence or absence of IL-4 and IL-2. Although limited, FDCs routinely supported B cell proliferation over baseline levels in three out of three experiments.

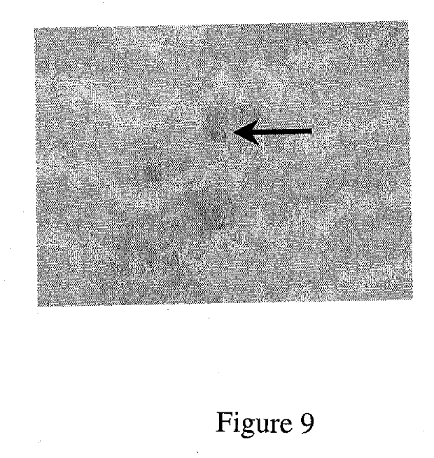

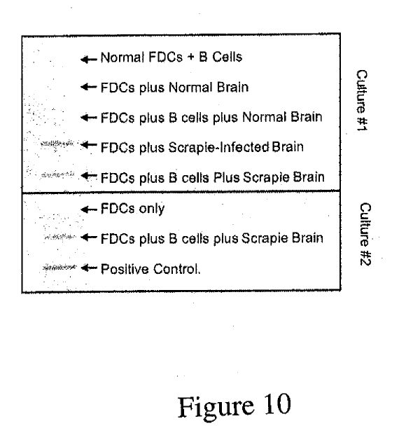

In preliminary studies, these cultures have been infected with PrP^{Sc}. Protocols shown to infect murine neuroblastoma cell lines with murine-adapted scrapie were adapted for our system. Of all conditions tested, those cultures incubated with both PrP^{Sc} and Scrapie-susceptible B cells appeared to show the best long-term infectivity in two out of two experiments. See FIGS. 9 and 10. FIG. 9 shows PrP^{Sc} in the cytoplasm of FDCs infected in vitro six weeks prior to analysis. FDCs were infected in the presence of peripheral blood B cells, and PrP^{Sc} homogenate was removed. Cells were cultured for an additional six weeks, and then analyzed by immunohistochemistry for PrP^{Sc} (indicated by the arrow).

### Example 4

A detailed protocol of prion infection of FDCs is as follows.

Overall Plan: Cells are serum-starved prior to and during infection. Although the infectivity is only carried out over a period of less than 24 hours, cells are then cultured up to several weeks to promote PrPSc propagation.

Preincubation of Homogenate: For each well to be infected, add 50 ul of 10% Brain homogenate to 50 μl of normal deer serum. Incubate at 37° C. for 1 hour prior to infection. 50 μl brain homogenate is diluted with 50 μl Media to a final volume of 100 μl per well.

Preparation of Cells: For PBMCs: Peripheral blood mononuclear cells from a CWD uninfected but susceptible animal are prepared using Percoll Gradients. Cells are counted, and resuspended at 108 cells/ml in Media for infection. For B cells, peripheral blood mononuclear cells from a CWD uninfected but susceptible animal are prepared using Percoll Gradients. Cells are counted, and resuspended at 108 cells/ml in PBS-1% FCS (1-2×108 cells total). 1 ml of antibody against CD4 (17D), CD8 (6-87), CD61 (1-44-19), and γδ-TcR (18-106) are added, and incubated for 10 minutes at 4 C. Cells are washed twice with PBS-FCS, and incubated with 200 ul goat anti-mouse-IgG magnetic beads per 108 cells at a final concentration of 108 cells/ml for 10 minutes at 4 C. Cells are washed 2×, and then negatively selected for B cells using the AutoMACS. Harvested cells are counted, and resuspended in media at 10-8 cells/ml for infection.
1) Plate FDCs in a 24-well culture dish. Grow to near confluence. For each infection:
a. Control
b. FDCs plus 100 μl diluted brain homogenate
c. FDCs plus 100 μl brain homogenate preincubated 1:1 with normal sheep serum
d. FDCs plus 100 μl diluted brain homogenate plus B cells (107/well)
e. FDCs plus 100 μl brain homogenate preincubated 1:1 with normal sheep serum plus B cells (107/well)
f. FDCs plus peripheral blood mononuclear cells (107/well) plus 100 μl diluted brain homogenate
g. FDCS plus peripheral blood mononuclear cells plus 100 μl brain homogenate preincubated 1:1 with normal sheep serum.
2) Remove media from FDCs, and wash cells twice with cold PBS.
3) Add 1.7 ml 1×HBSS containing 10% FCS to each well. Incubate 1 hour at 37° C.
4) Add 107 cells to those wells requiring cells (total volume not to exceed control)
5) Add 100 μl of Brain homogenate, appropriately treated (i.e. preincubated or not).
6) Incubate overnight at 37° C.
7) Wash cells 2× with PBS. Discard as BIOHAZARDOUS and treat with bleach prior to disposal.
8) Add 2 ml IMDM/10% FCS containing 106 B cells sorted as described above, and continue to culture as normal, treating all tissue culture supernatant as contaminated material.
9) Freeze several aliquots of each for future experiments over the next 4-6 weeks (Freeze in 10% DMSO/90% FCS).
10) At 4, 7, 10, and 14 days post-infection, prepare cytospins for analysis by immunohistochemistry using mAb 15B3 to detect PrPSc expression and lyse cells for slot-blot analysis.

### Example 6

A detailed Protocol for the isolation of B cells is as follows. Peripheral blood mononuclear cells from a scrapie-uninfected but susceptible animal are prepared using Percoll Gradients. Cells are counted, and resuspended at 108 cells/ml in PBS-1% FCS (1-2×108 cells total). 1 ml of antibody against CD4 (17D), CD8 (6-87), CD61 (1-44-19), and γδ-TcR (18-106) are added, and incubated for 10 minutes at 4° C. Cells are washed twice with PBS-FCS, and incubated with 200 μl GAM-IgG magnetic beads per 108 cells at a final concentration of 107 cells/ml for 10 minutes at 4° C. Cells are washed 2×, and then negatively selected for B cells using the AutoMACS. Harvested cells are counted, and resuspended in media containing 100 ng/ml E. Coli lipopolysaccharide (LPS) at 10-7 cells/ml for infection.
1) Plate FDCs in a 24-well culture dish. Grow to near confluence. For each animal, prepare 8 wells for infection (duplicates at each time point). Each pair of wells will be used for a different time point, such that replication of PrP^{CWD} may be assessed 4, 7, 10, and 14 days after inoculation.
2) Remove media from FDCs, and wash cells twice with media.
3) Add 107 cells to each well.
4) Incubate at 37° C. Add fresh media each 4 days, being careful not to disturb adherent B cells.
5) At 4, 7, 10, and 14 days after infection, remove media from 1 well, and fix cells in acetone. Stain cells directly on the plate using mAb 15B3 followed by Alexa-Fluor 488 conjugated Goat-Anti-Mouse IgM for detection by immunofluorescence.
6) At 4, 7, 10, and 14 days after infection, remove media and harvest all cells by trypsinization. Recover cells by centrifugation, and analyze for PrP^{CWD} proliferation by slot blot.

FIG. 10 shows PrP^{Sc} in FDCs infected in vitro two weeks prior to analysis. FDCs were infected as described in the figure, and PrP^{Sc} homogenate was removed. Cells were cultured for an additional two weeks, and a proteinase-K treated cell lysate of each culture was analyzed by slot blot according to established protocols. Two separate experiments are shown in FIG. 10.

Simply put, FDCs were required to support B cell growth, and B cell growth was required to propagate the prion protein. Therefore, both FDCs and B cells are required to propagate the PrP^{Sc} in vitro. The FDCs also serve to “concentrate” the PrP^{Sc}, as only a subset of FDCs appeared to be positive for PrP^{Sc} six weeks after inoculation. These data would indicate that long-term FDC cultures possess the capability to retain and potentially propagate PrP^{Sc} in vitro. The utility of the FDC culture technique for diagnosis of blood samples from infected animals was then assessed, and ante mortem tests were developed.

### Example 5

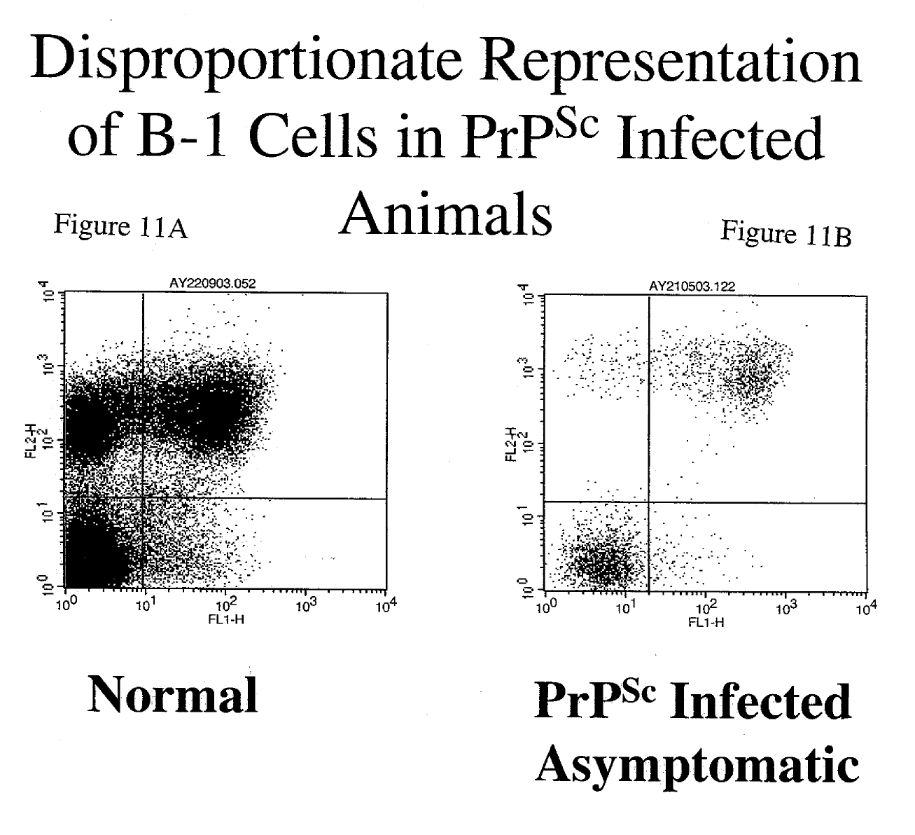

Peripheral blood B cells was isolated from two sheep, one of which had been infected two months previously with an intracerebral injection of scrapie brain homogenate. Given that the normal incubation for this isolate ranges from 14 to 17 months, it seems likely that only a limited number of B cells would be available potentially affected with PrP^{Sc}. Nonetheless, B cells from peripheral blood were plated on cultured FDCs, and co-cultured for 10 days. No exogenous PrP^{Sc} was seeded into the culture. Following incubation, an antibody specific for the pathogenic prion protein (15B3, obtained for research purposes from Prionics, Inc) was used to stain the cultures for the presence of PrP^{Sc}. Cultures from the infected animal were strongly positive using standard immunofluorescence, whereas, those obtained from the uninfected animal were negative. See FIGS. 11A and 11B, which show immunofluorescence staining of FDC cultures ten days after initiation of co-incubation with B cells from an uninfected (left) and scrapie-infected (right) sheep. Note the cells strongly staining with the PrP^{Sc} specific monoclonal antibody 15B3 in the right panel (arrow). Only diffuse, nonspecific staining is evident in the cultures from the uninfected animal.

The phenotype and composition of the peripheral blood B cell pool in 10 Scrapie-infected and 10 uninfected age-matched animals was tracked. During sequential analysis, we found a trend for over-representation of B-1-like cells in the peripheral blood of Scrapie-infected animals. See FIGS. 11A and 11B, which show that B-1-like cells expressing CD11b are disproportionately represented in the peripheral blood of Scrapie-infected animals (Y-axis, B cell CD72 marker, X-axis, CD11b).

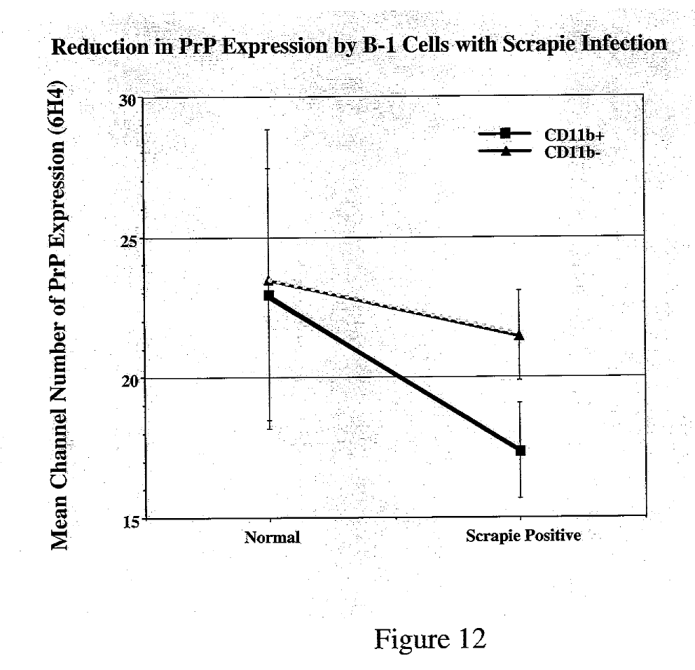

Although there were no significant differences in the overall number of peripheral blood B cells, there was a shift towards greater representation of B-1-like cells associated with disease. Surprisingly, there was also a significant reduction in the expression of PrP^{C} on B cells associated with progression of diseases. See FIG. 12, which shows the reduction in PrP^{C} expression on B-1 cells during scrapie progression. PrP^{C} expression was monitored on the surface of B-2 cells (top line) and B-1 cells (bottom line) using 6H4 mAb over the course of Scrapie progression.

Specifically, there was a statistically significant reduction in PrP^{C} expression on the surface of B-1-like cells collected from the peripheral blood of scrapie infected animals. Taken together, these data may suggest a prion-induced shift in the differentiation of B-1-like cells in the lymph nodes of Scrapie-infected animals. Our working hypothesis, central to this proposal, is that Scrapie infection results in selective deletion of B-2-like cells in affected germinal centers, and selection for PrP^{C}-low B-1-like cells. While this shift does not appear to have significant effects on overall immune competence, we believe it reflects local events occurring in affected germinal centers.

### Example 6

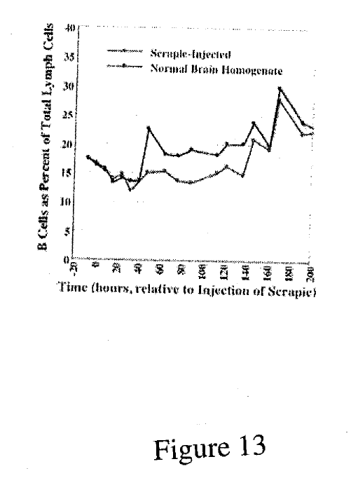

B cell subsets in acute prion disease were analyzed. PrP^{Sc} is likely transported via migratory leukocytes from initial sites of infection to FDCs in lymph nodes. Once there, PrP^{C} proliferates on concentrates through interaction with affected FDCs, where it is then transferred to regional proliferating B cells and Tingible Body Macrophages via iccosomes. The overall implication of these studies is that PrP^{Sc} should selectively inhibit B cell development in affected lymph nodes. To test the regional response of lymph nodes to infection with PrP^{Sc}, we cannulated efferent lymphatics draining bilateral prefemoral lymph nodes. As lymph drains into these two lymph nodes from unique tissue beds, it is possible to selectively inoculate one lymph node with a test material (PrP^{Sc}) while reserving the contralateral lymph node as a control. Using this methodology, we injected 200 μl of a 10% brain homogenate from a Scrapie positive animal into the drainage area of the right prefemoral lymph node, and an equal volume of 10% brain homogenate from a normal animal into the left side. Efferent lymph was then collected at regular intervals over the next 10 days, and phenotyped to determine changes in the output of specific cell types which reflects the ongoing immune response in the local lymph node. While there were equivalent changes in the overall cell output and output of CD4 and CD8 positive T cells from both lymph nodes, there was a significant reduction in the output of B cells from the Scrapie-injected side. See FIG. 13, which shows the reduction in B-cell output from Scrapie-inoculated lymph nodes. Following injection of Scrapie-infected brain homogenate, there is a transient but significant reduction in the output of B cells in the regional lymph. Top blue line=normal brain; Bottom red line=Scrapie brain.

While it is possible that this reduction in cell output associated with local scrapie stimulation could be explained by an induced selective retention of B cells within the lymph node, these observations would also be consistent with a selective inhibition of B cell proliferation within the Scrapie-injected lymph node. These possibilities can be differentiated using an in vitro model of FDC-B cell interactions of Scrapie-affected germinal centers.

### Example 7

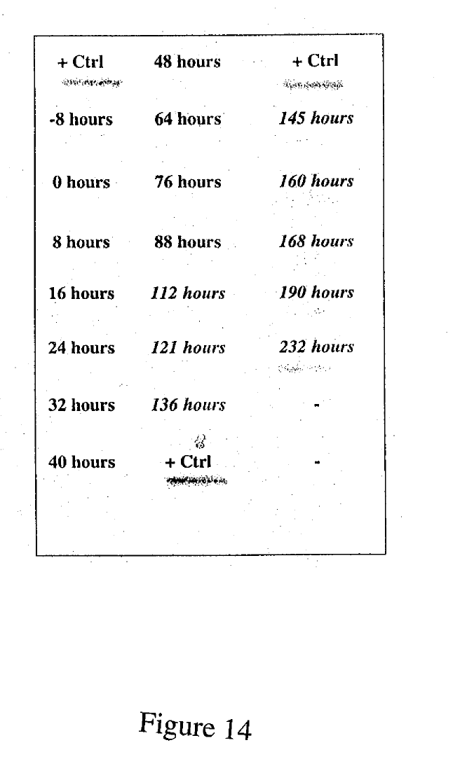

Transport of prions by migratory B cells was investigated. Although it has been known for some time that blood can effectively transmit prion disease, the nature of the infectious particle remains in question. Given recent data that suggests that migratory B cells may transport infectious prion protein, we collected efferent lymph cells and efferent lymph plasma draining a lymph node acutely infected with scrapie as described above. Although samples of efferent lymph plasma routinely tested negative from both Scrapie-injected and control lymph nodes, cells testing positive for PrP^{Sc} could be found draining only the Scrapie-injected lymph node by both immunohistochemistry and dot-blot. See FIG. 14. Intriguingly, the concentration of cell associated PrP^{Sc} appeared beginning approximately 5 days after local Scrapie injection, and continued to increase until the experiment was terminated 10 days following injection. Although it is clear that migratory leukocytes are capable of transporting PrP^{Sc} from affected lymph nodes as demonstrated in 3 independent experiments, further experiments are necessary to confirm this data and confirm that B cells are the cell type necessary for this transport.

FIG. 14 shows PrP^{Sc}-laden lymphocytes exit the lymph node beginning 136 hours after injection, traveling via the lymph to the systemic circulation. Lymphocytes were harvested from lymph, washed three times, and 10-million cells harvested for analysis by slotblot for PrP^{Sc} expression. Diluted Scrapie-brain homogenate was used as a positive control. Note that PrP^{Sc} increases in the cell-bound fraction until the termination of the experiment 232 hours after injection. Afferent lymph cells leaving a scrapie-injected site were also found to contain PrP^{Sc}, however peak recovery of these cells occurred within the first 24 hours of infection (not shown).

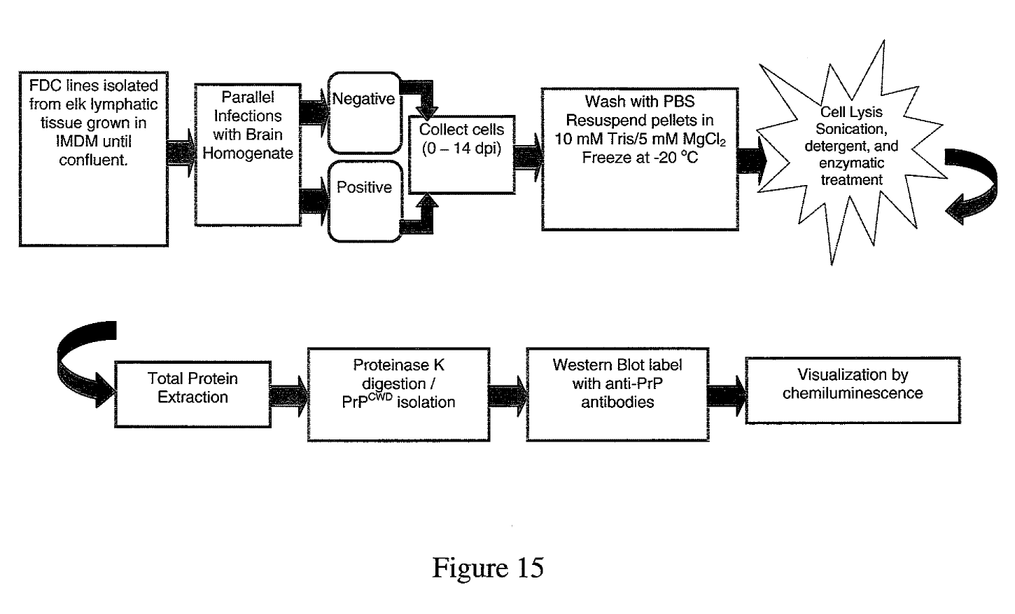

The isolation of PrP^{CWD} and Western blot analysis is illustrated in FIG. 15. The ability of isolated FDC cultures to mimic scrapie-infected germinal centers was tested. Sheep FDC line 6A was infected with 200 μl of 10% scrapie-brain homogenate on day 0, and washed extensively on day 1 to remove the initial inoculum. Aliquots of cells were collected 4, 7, and 14 days after scrapie infection, at which point the infected cell cultures were split 1:3 and cultured to confluence; At each successive passage, samples were collected and analyzed by a PrP^{Sc} enrichment Western Blot for the presence of PrP^{Sc}, and remaining cells passaged 1:3 over a period of approximately three months, and successive cultures analyzed by Western Blot for the presence of protease-K resistant Prion protein (PrPSc). See FIG. 16. PrPSc is clearly evident through the 3rd blind passage. Cultured FDCs would remain PrP^{Sc} positive for greater than 4 passages (i.e. >10 weeks) following initial scrapie infection. These results indicate that FDC cultures possess the ability to be infected, maintain and potentially propagate PrP^{Sc}, and support B cell proliferation in vitro.

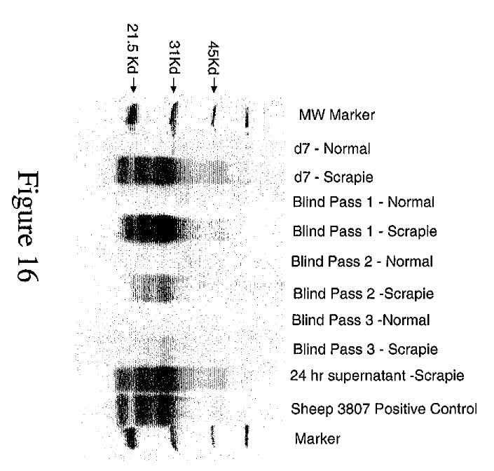

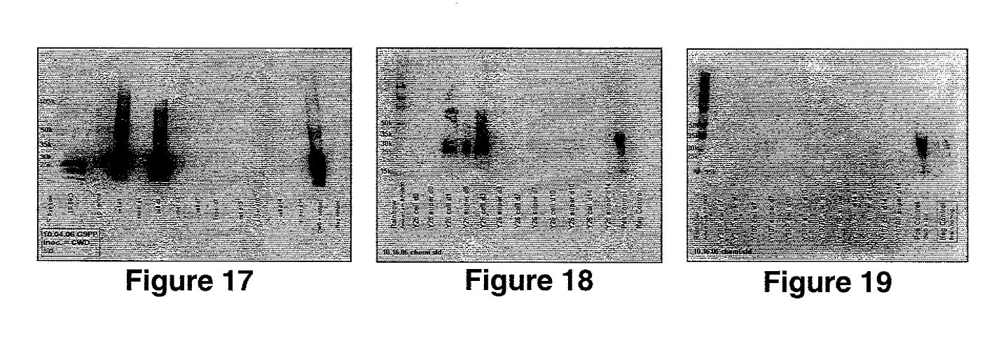

FIGS. 17-19 show Western blots of elk FDC lines infected with CWD-positive brain homogenate. FIG. 17 shows Peyer's Patch-derived elk cell line G9. FIG. 18 shows mesenteric lymph node-derived elk cell line Y22. FIG. 19 shows retropharyngeal lymph node-derived Y3 and Y107. Time points from day 7 through day 14 (days post infection) are shown.

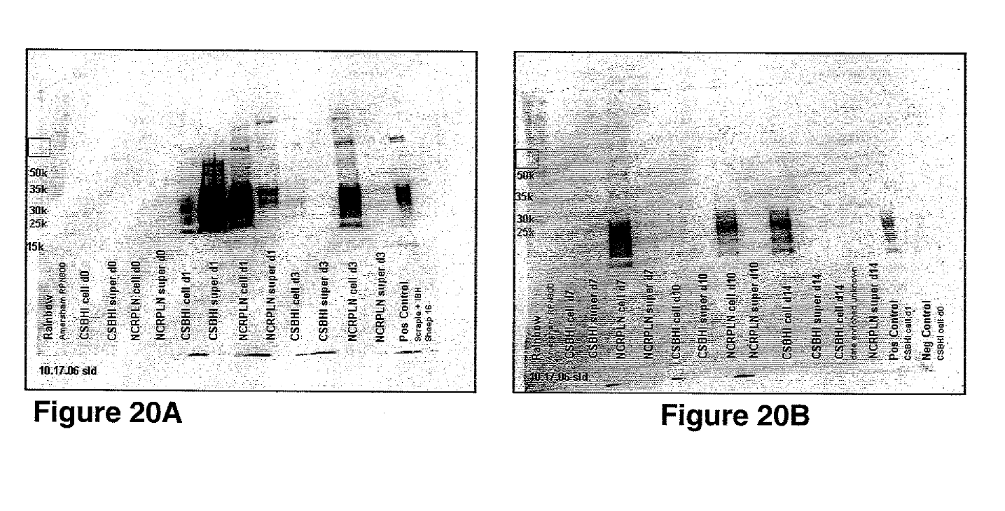

FIGS. 20A and 20B show Western blots of cattle FDC lines infected with sheep scrapie. Cattle FDC lines were prepared from lymph nodes and ileal Peyer's patches and infected with a 10% homogenate of sheep scrapie-infected brain. Cell-associated scrapie protein could be detected up to 14 days following infection in lines prepared from both retropharyngeal lymph nodes and ileal Peyer's patches. This demonstrates that the in vivo species specificity for infection of FDCs with prions is not evident in vitro.

The invention has been described with reference to various specific and illustrative embodiments and techniques. However, it should be understood that many variations and modifications may be made while remaining within the spirit and scope of the invention.

We claim:

1. A method for in vitro propagation of infectious prions (PrP^{Sc}) comprising:
providing a culture of follicular dendritic cells (FDC);
adding infections prions to the FDC culture; and
culturing the infected cells.

2. The method of claim 1, further including the step of adding peripheral B cells to the FDC culture to obtain a combined cell culture.

3. A method of detecting infectious prions (PrP^{Sc}) in an animal or human comprising:
collecting peripheral blood B cells from an animal or human suspected of being infected with infections prions;
co-culturing the B cells with cultured follicular dendritic cells; and
detecting infectious prions by a specific binding assay.

4. The method of claim 3, wherein the specific binding assay is an immunological assay.

5. The method of claim 4, wherein the immunological assay includes immunohistochemistry.

6. The method of claim 5, wherein said immunohistochemistry includes Western blots.

7. The method of claim 4, wherein said animal is an ovine, and the immunological assay involves an antibody specific for scrapie.

8. The method of claim 4, wherein said animal is a cervid, and the immunological assay involves an antibody specific for Chronic Wasting Disease (CWD).

9. The method of claim 4, for detection of infectious prions in a human, wherein the immunological assay involves an antibody that binds human prion protein (PrP).

10. The method of claim 4, for detection of infectious prions in a bovine, wherein the immunological assay involves an antibody that binds bovine prion protein (PrP).

11. A method of detecting infectious prions (PrP^{Sc}) in an animal or human comprising:
obtaining a fluid, cellular or tissue sample from an animal or human suspected of being infected with infections prions;
adding said sample to a culture of follicular dendritic cells and culturing said cells; and
detecting infectious prions in said culture by specific binding assay.

12. The method of claim 11, wherein said culture of follicular dendritic cells includes B-cells.

13. The method of claim 11, wherein said specific binding assay is an immunological assay.

14. The method of claim 13, wherein said immunological assay includes immunohistochemistry.

15. The method of claim 11, wherein said sample is selected from the group consisting of blood, brain, spleen, spinal fluid, lymph nodes, and tonsils.

16. A method for in vitro propagation of infectious prions (PrP^{Sc}) comprising:
selecting an animal susceptible to a prion disorder;
obtaining lymph node cells from said animal;
selecting lymph node cells that bind antibodies specific for FDCs and culturing the resulting cells;
selecting cells from the culture that bind antibodies specific for prion protein;
infecting said selected cells with infections prions, and
culturing said infected cells.

17. The method of claim 16, wherein said step of selecting an animal involves selecting an animal genetically susceptible to a prion disorder.

18. The method of claim 17, wherein said animal is an ovine and said prion disorder is scrapie.

19. The method of claim 17, wherein said animal is a cervid and said prion disorder is Chronic Wasting Disease (CWD).

20. The method of claim 16, wherein said animal is a bovine and said prion disorder is bovine spongiform encephalopathy.

21. A method of detecting, and optionally quantifying, prion in a biological sample, said method comprising:
contacting said biological sample with a mixed culture of FDCs and B-cells, under conditions that allow the infection thereof; and
detecting infection or non-infection of the cultured cells, wherein the presence of infection is indicative of prion in the sample.

22. The method of claim 21, wherein said sample is selected from the group consisting of blood, lymph node, and brain.

23. The method of claim 21, wherein said mixed culture of FDCs and B-cells include cells isolated from an animal genetically susceptible to prion disease.

24. The method of claim 21, wherein said detecting includes an immunological assay.

25. A kit for detecting infectious prions (PrP^{Sc}) in a biological sample, the kit comprising:
cultured follicular dendritic cells (FDCs);
antibodies specific for infectious prions (PrP^{Sc}).

26. The kit of claim 25, further comprising B cells co-cultured with the FDCs.

27. The kit of claim 25, wherein said FDCs are cervid and the antibodies specifically bind Chronic Wasting Disease (CWD).

28. The kit of claim 25, wherein said FDCs are ovine and the antibodies specifically bind sheep Scrapie.
